(19) United States
(12) Patent Application Publication
(10) Pub. No.: US 2004/0132103 A1
(43) Pub. Date: Jul. 8, 2004

(54) Process and apparatus for the combinatorial preparation of mixtures, and use of these

(76) Inventors: Michael Haubs, Bad Kreuznach (DE); Thomas Reisinger, Ingelheim (DE); Arnold Schneller, Messel (DE)

Correspondence Address: CONNOLLY BOVE LODGE & HUTZ, LLP, P O BOX 2207, WILMINGTON, DE, 19899, US

(21) Appl. No.: 10/688,615

(22) Filed: Oct. 17, 2003

(30) Foreign Application Priority Data
Oct. 18, 2002 (DE) DE 102 48 639.5

(51) Int. Cl. G01N 33/53; G01N 33/543

(52) U.S. Cl. 435/7.1; 436/518

(57) ABSTRACT

The present invention relates to a continuous process for the preparation of mixtures from a wide variety of components, and also to an apparatus for carrying out the process. The process encompasses the steps of:

a) charging the individual components to storage vessels,

b) introducing each individual component by way of a conveying device for this component into a mixing device,

c) varying the conveying rate of at least one conveying device in such a way that this conveying rate varies periodically between a lower and an upper limiting value, and

d) mixing the individual components in the mixing device.

An example of an application of the invention is the preparation of substance libraries for high-throughput screening in the plastics industry, in particular of mixtures of polymers with one another, and of mixtures of polymers with additives.

The present invention relates to a process for the combinatorial preparation of mixtures of chemical compounds, for example for the preparation of plastics mixtures suitable for high-throughput screening, and to an apparatus suitable for carrying out this process.

A number of automatic apparatuses have been developed for preparing mixtures of chemical compounds. For example, U.S. Pat. No. 4,595,496 describes an apparatus for the controlled feed to an apparatus for liquid chromatography, in which the liquids to be applied are charged to individual storage devices and are fed by way of supply lines and a pump to the chromatography column. The feed takes place by means of a pump. In each supply line there is a valve which is activated selectively and periodically. This permits attenuation of the variations brought about by the operation of the pump in the conveying rate of the mixture fed.

The use of highly automated combinatorial methods to test the activity of substances is a well-established constituent of research in the sectors of pharmaceuticals and plant protection. The expression “combinatorial methods” generally refers to the production of a large number of chemically different compounds or mixtures and the subsequent rapid testing of these substance libraries for one or more properties. The synonymous term high-throughput screening is also used for these methods, because, alongside other advantages, they especially permit a marked increase in the speed of sample throughput. For example, use of these methods permits the activity of some tens of thousands of substances to be checked every day in searches for active ingredients. Examples of the use of combinatorial methods are given by Lowe, J C S Reviews, 309-317 (1995), 20 N. K. Terreft, Combinatorial Chemistry, Oxford University Press, Oxford, 1998, Combinatorial Chemistry and Molecular Diversity in Drug Discovery (eds.: E. M. Gordon, J. F. Kerwin), Wiley, New York, 1998.

Recently, these methods of combinatorial chemistry and high-throughput screening have received increasing attention in materials science, for example in the development of materials with optical uses, or the discovery of new catalysts. An example of an overview of these relatively new developments is found in the article by B. Jandeleit, D. J. Schäfer, T. S. Powers, H. W. Turner, W. H. Weinberg in Angewandte Chemie 1999, 111, 2648-2689.

Combinatorial methods have hitherto been very little used in research and development in the formulations sector, particularly in polymer formulations.

The approaches described hitherto for the production and testing of substance libraries, including those for polymers or polymer formulations, are based on discrete, spatially separate containers (compartments) in which the mixtures are produced and then tested.

U.S. Pat. No. 5,985,356 describes the production and screening of various inorganic or organic materials. It also describes the copolymerization of styrene with acrylonitrile in toluene in an arrangement composed of compartments of size 3×3×5 mm. This requires complicated apparatuses for precise metering of monomers and initiator.

WO-A-99/52,962 describes a method for preparing alternating copolymers. In this, by way of example, the diol component and, respectively, the dicarboxylic acid components are varied systematically in an arrangement of 8 times 14 reaction vessels, and the resultant copolymers are studied for selected properties.

WO-A-00/40331 describes an apparatus and a combinatorial method for the discovery of catalysts and polymers. It uses an apparatus for polymerizing monomers in reactors arranged in parallel.

A discussion paper from the National Institute of Standards and Technology (M. R. Nyden, J. W. Gilman, Proceedings, Fire Retardant Chemicals Association, Mar. 12-15, 2000, Washington, D.C., 1-5 pp. 2000) mentions the continuous production of polymer formulations. (Internet address: http:/fire.nist.gov/bfrlpubs/fire00/PDF/f00017.pdf).

That publication discusses a process for continuous production and testing of polymer formulations with flame retardants, proposing for that purpose a system composed of a computer-controlled gravimetric solids feed and an extruder which is not specified in any further detail. The arrangement is intended to extrude polymers with flame-retardant additives in concentrations programmed in advance, these then being analyzed on-line and tested for fire performance.

The variation in concentration of the flame-retardant additive is intended to take place deterministically by way of the computer-controlled gravimetric feed unit in the previously-set concentration steps, without covering the entire phase space.

A phase space includes all of the theoretically possible compositions of a multicomponent system. It can be represented as a multidimensional space with orthogonal coordinates which give the concentrations of the components making up the mixture. In the case of a mixture of, for example, five components, a point in the five-dimensional phase space is unambiguously defined by way of the data for the concentrations of the five components.

There is a limit to the precision with which phase spaces can be depicted. Because any multicomponent system has an infinite number of compositions, practical operations have to be carried out with limited compositional resolution. The higher the desired compositional resolution, the greater the number of different-concentration mixtures that have to be produced.

None of the processes known hitherto covers the entire phase space or selected portions of the phase space at a prescribed level of resolution during the preparation of mixtures.

It is an object of the present invention to provide a process of this type and an apparatus suitable for carrying out the process.

A further object of the present invention consists in providing a process for the high-throughput screening of multicomponent formulations (at least two components).

A further object of the present invention consists in the provision of a process which can, in a simple manner, set the compositional resolution of mixtures as desired, in order to minimize the cost for the apparatus and time needed for a given task.

The invention provides a process for the continuous preparation of mixtures from at least two components, encompassing the steps of:

a) charging the individual components to storage vessels,

b) introducing each individual component by way of a conveying device for that component into a mixing device,

c) varying the conveying rate of at least one conveying device in such a way that this conveying rate varies periodically between a lower and an upper limiting value, and

d) mixing the individual components in the mixing device.

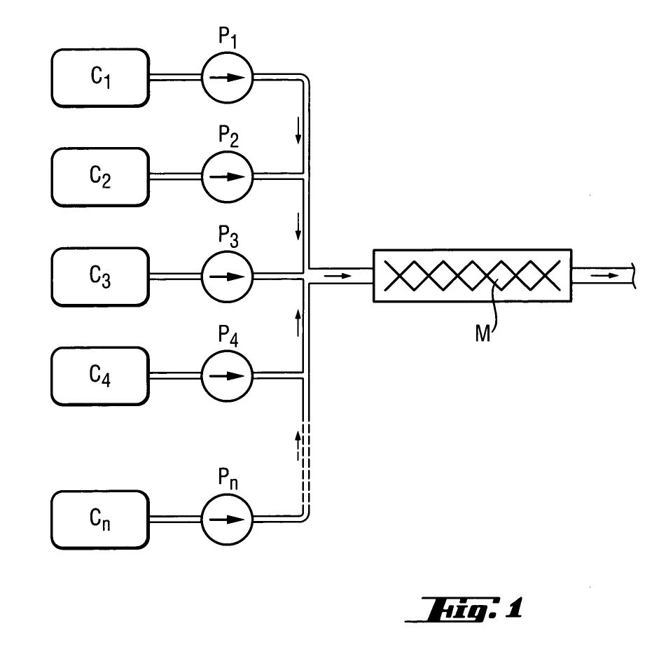

The achievement of the abovementioned objects is described by way of example for a system with n components, using the diagram shown in FIG. 1.

Components 1 to n are present in the storage vessels C₁ to Cₙ, component i being in vessel Cᵢ. Each vessel Cᵢ has been connected by way of a conveying apparatus (e.g. a pump) Pᵢ to a mixing device (hereinafter also termed mixer M). The finished multicomponent mixture may be further used at the outlet from the mixer.

To cover the entire phase space, the conveying rate CR(t) (CR(t)=dV/dt) [V=volume conveyed per unit of time; t=time] of the individual conveying devices (e.g. pumps) is controlled as a function of time. All of the conveying devices here are individually controllable and may follow different conveying rate/time functions CR(t). The nature of the periodic conveying rate/time function CR(t) may be that of any desired periodic function, or else may be a constant, but at least one of these conveying rate/time functions CR(t) has to be periodic, and at least one of these conveying rate/time functions is non-pulsed (CR(t)<∞).

The process of the invention permits the entire phase space of a mixture of prescribed components to be covered with any desired prescribed precision.

At least the conveying rate of one conveying device is varied periodically. The conveying rates of two or more conveying devices are preferably varied periodically, the frequencies of the variations differing from one another.

Particular preference is given to a process in which the variation of the conveying rate of one conveying device continuously rises or falls, and wherein the variation in the conveying rate of all of the other conveying devices is periodic.

Particular preference is also given to a process in which the variation of the conveying rate of at least one conveying device, preferably of all of the periodic variations, corresponds to a sawtooth function or a sine function, the periods thereof preferably being constant over time.

Very particular preference is given to a process in which the variation of the conveying rate of at least one conveying device corresponds to a periodic step function whose periods and step intervals are preferably constant over time.

In another, particularly preferred, version of the process of the invention, the periods or step intervals for the variation of the conveying rates of the individual conveying devices are an integral multiple of a base period, where the ratio of any two desired periods or step intervals for the variation of the conveying rate of the conveying devices, or the ratio of a period and a step interval for the variation of the conveying rate of two conveying devices, is preferably equal to half of a whole number, and is in particular 0.5, or 1.5, or 2.5.

The periods or step intervals for the variations of the conveying rates of the individual conveying devices are preferably an integral multiple of a base period, and the minimum period or step interval may be selected as desired.

The periods or step intervals for the variation of the conveying rates of the individual conveying devices are preferably held constant over time.

The phase shifts of the periods or of the step intervals for the variation of the conveying rates of the individual conveying devices are preferably held constant over time.

The phase shifts of the periods or step intervals of the conveying rates of the individual conveying devices are very particularly preferably held equal to zero.

The following process measures have proven particularly successful and may be used individually or in a combination in one or more of these measures:

A) one conveying device is operated in continuously rising or falling mode;

B) all of the other conveying devices follow periodic functions;

C) the frequencies of the conveying apparatuses for the individual components differ from one another;

D) the ideal function for a conveying device is a sawtooth function or a sine function;

E) the ideal functions for all of the other conveying devices are periodic step functions;

F) the periods and step intervals are preferably constant over time;

G) the ratio of any two desired periods or step intervals, or the ratio of a period and a step interval, is preferably half of a whole number (e.g. 0.5, or 1.5, or 2.5);

H) the frequency ratio is proportional to the desired compositional resolution;

I) all of the periodic functions preferably start with the minimum conveying rate CR(t=0)=CRₘᵢₙ;

J) the phase shift of any two desired periodic functions with respect to one another may likewise be freely selected (but is preferably equal to zero in the case of the step functions);

K) the maximum conveying rate of the conveying apparatuses (amplitude of the periodic function), in relation one to the other, depends on the desired compositions;

L) the resolution of the compositions is proportional to the number of concentrations set between the minimum for the metering method and the maximum value.

The minimum frequency for the periodic conveying rate/time functions CR(t) may be selected as desired, whereas the maximum frequency depends on parameters of the apparatus, of the mixing components, or of the mixture, for example on the substance to be metered, on the conveying device, and on the axial dispersion in the mixer.

In another preferred method of operation, the total conveying rate of all of the conveying devices is constant over time.

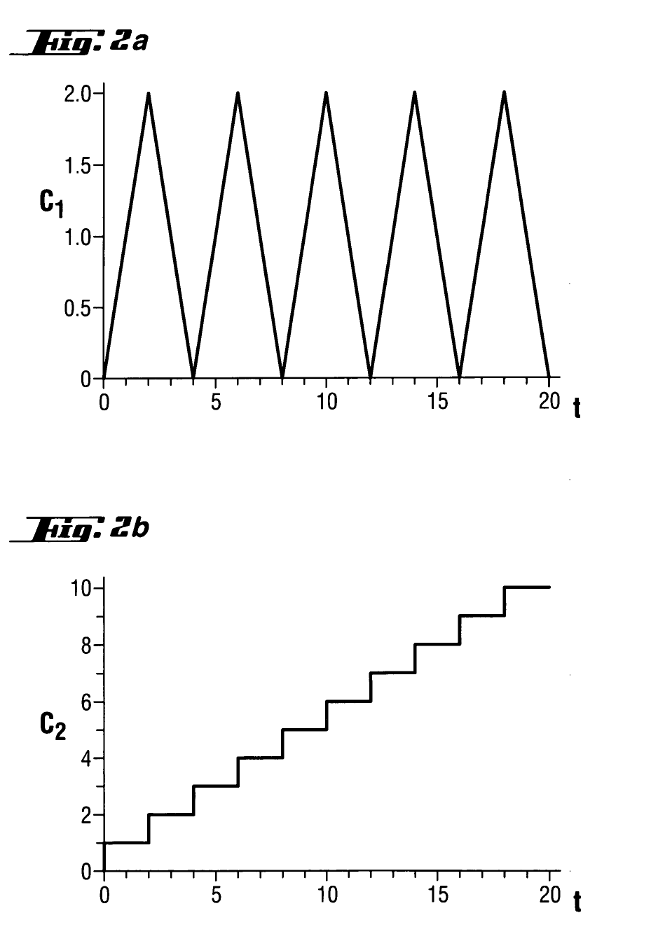

FIGS. 2a and 2b illustrate these preferred procedures set out in items A) to L). The conveying rate/time functions CR1(t) [FIG. 2a)] and CR2(t) [FIG. 2b)] are shown for two components, the time (in unspecified units) being plotted on the abscissa and the concentrations C₁ and C₂, respectively, of components 1 and 2 (in unspecified units) being plotted on the ordinate.

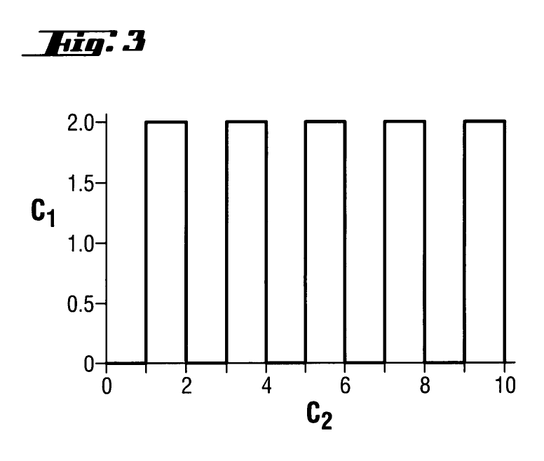

This gives the phase space diagram shown in FIG. 3, where abscissa and ordinate, respectively, show the concentration C₁ and C₂ of components 1 and 2, respectively, in the resultant composition.

The process of the invention permits any desired mixtures to be prepared from any desired conveyable substances, preference being given to mixtures of liquids, conveyable solids, and/or gases.

It is preferable to prepare mixtures from fluid solids, and/or polymer melts, and/or masterbatches.

Examples of components of the mixtures to be prepared are any of the inorganic or organic materials which may be bonded by any desired bonds, for example by ionic bonds, covalent bonds, or by complexing.

Examples of inorganic materials are metals, semimetals, or metal alloys, or metal salts, or else the oxides, sulfides, sulfites, sulfates, phosphates, or halides of metals or of semimetals.

Components of the mixtures to be prepared may also be ceramics.

Examples of organic materials are compounds whose main constituents are carbon and hydrogen and in which, where appropriate, relatively small proportions of oxygen, nitrogen, phosphorus, and/or other elements are also present.

These may be biological materials, or in particular non-biological materials. Besides low-molar-mass compounds, for example with molar mass up to 500 g/mol, use is made of high-molecular-weight compounds, in particular polymers. Besides the traditional organic materials, use may also be made of organometallic materials.

The components of the mixtures to be prepared may have any desired properties, examples being electrical conductors (including superconductors), semiconductors, or insulators, and/or thermal conductors or insulators, or may have diamagnetic, paramagnetic, or ferromagnetic properties.

The process of the invention can also meter two or more additives simultaneously in varying concentration into a screening experiment. For example, simultaneous variation of the concentration of the additives can generate a substance library.

The mixtures prepared by the process of the invention may encompass a partial volume (where appropriate with a relatively high number of dimensions) of the phase diagram of a multicomponent mixture. These are therefore suitable for wide-ranging high-throughput screening. It is possible here to encompass concentration ranges smaller than 1% for individual constituents of a mixture.

To carry out high-throughput screening, the mixtures prepared combinatorially in the mixing assembly may be continuously converted to a form amenable to further processing and testing.

One advantageous variation of the invention is a process for the continuous preparation of mixtures from at least one thermoplastic polymer and at least one additive, wherein at least one thermoplastic polymer is fed continuously or in a succession of pulses to a mixing assembly, melted, and mixed with one or more additives, one or more additives being thus fed to the mixing assembly in one or more successions of pulses, the polymer mixture is continuously discharged from the mixing assembly, and is transformed into a form amenable to further processing and testing.

Another advantage of the mixtures prepared by the present invention is that the continuously prepared product can readily be divided into discrete fractions of any desired size, whereas the processes of the prior art can, by virtue of the process itself, only give discrete fractions whose properties have to be planned individually prior to carrying out the experiment, and which are not transformable into a continuous stream of product, even when that would be advantageous for certain investigation methods.

The invention also provides an apparatus for the mixing process described above.

The apparatus of the invention has:

i) storage vessels for each individual component of the mixture to be prepared,

ii) mixing device for mixing all of the components of the mixture to be prepared;

iii) lines for the individual components, leading from each individual storage vessel to the mixing device;

iv) in every line for every individual component, conveying devices whose conveying rate can be set individually; and

v) control device for the conveying devices, which controls the conveying rate of each conveying device independently of the others, and which sets the conveying rate of at least one conveying device variably and periodically between a predetermined lower limiting value and a predetermined upper limiting value.

The achievement of the abovementioned objects is described by way of example for a system with n components, using the diagram shown in FIG. 1.

Any desired mixing assembly may be used to prepare the mixture of the components.

In one particularly preferred embodiment, this is a continuous mixer.

Static mixers are suitable.

In one preferred embodiment of the process of the invention, the mixing assembly is composed of at least one screw machine.

In one preferred embodiment, the screw machines used are extruders, particularly preferably twin-screw extruders.

The conveying devices serve to feed the mixing assembly with components of the mixture to be formed, for example in the form of powder or liquid or pellets, either in pure form, or premixed in masterbatches.

The feed of the component(s), for example of polymers and, where appropriate, of other additives, takes place continuously.

The metering methods of the prior art may be used for the process of the invention, for feeding the individual components to the mixing assembly. A comprehensive description of metering systems used in industry was published in 1989 in “Dosieren von Feststoffen (Schüttgütern) [Metering of (bulk) solids]” from the company Gericke. Supplementary to that publication, the VDI report “Kunststoffe im Automobilbau” [Plastics in automotive construction], Vol. No.: 4224(2000) includes an up-to-date section concerning the metering systems usually used. These publications are incorporated by way of reference.

Within the metering process, a distinction is made between the single-stream metering process and the multistream metering process.

In the single-stream metering process, the polymers are metered into the main inlet of the mixing assembly together with the additives. For this, use is made of feed hoppers and/or ancillary input equipment with horizontal or vertical screws.

The multistream metering process is also termed fractionated metering or the split feed technique. Here, various constituents are added separately.

A distinction is also made between volumetric metering and gravimetric metering.

In the case of volumetric metering, appropriately designed screws for pellets, powder, fiber, and chips have what are known as decompactors, as required by the flow behavior of the bulk material. Besides screws, vibrating troughs or belt metering systems are also used for the volumetric metering of pellets, coarse-grained powder, fibers, or flakes.

Gravimetric metering equipment used comprises velocity-regulated and weight-regulated metering belt weighers, metering screw weighers, differential metering weighers with screw or vibrating trough, and quasi-continuous hopper weighers.

The annular groove metering system is used for volumetric or gravimetric metering of very small amounts of powder (about 10 g/h), this being where screw metering systems fail. Liquid constituents are fed to the mixing assembly through, for example, volumetric metering pumps.

If the metering pumps are regulated by means of a differential weigher, gravimetric metering is also possible for the addition of liquids.

Another possibility is pulsed or ramped addition of additives by way of other metering units.

By way of example, an ejector weigher is used for pulsed addition.

In the metering process, a distinction is made between gravimetric and volumetric addition.

The control device used for the conveying devices for the independent regulation of the conveying rate of each conveying device, and for setting the periodically varying conveying rate of at least one conveying device between a predetermined lower limiting value and a predetermined upper limiting value may be a data-processing system which is conventional per se, for example an appropriately programmed computer.

The invention also provides the use of the process of the invention for preparing substance libraries for high-throughput screening and other combinatorial methods.

For this, it is preferable to produce moldings from mixtures by the process of the invention, preferably in the form of film strips, extrudates, or pellets produced from these extrudates.

The mixture is preferably present in the form, for example, of an extrudate or of an unsupported film strip, so that these can easily be converted, for example by chopping or stamping of the film strip, or pelletization of the extrudate, into discrete fractions if this is advantageous for the subsequent processing or investigation.

The mixture prepared may be exposed for a certain period or over a certain distance downstream of the mixing assembly to a defined environment or treatment or treatment pathway.

In this process, the mixture may be exposed to certain temperature and humidity conditions, to a temperature profile, to one or more liquids, to moisture, to one or more gases, to one or more solids, or to mixtures of liquids and gases and solids, or to one or more types of electromagnetic radiation.

In this context, liquids or solids may be any of the organic or inorganic liquid and/or solid substances and/or biological living matter or substances. Another possible treatment is a mechanical load.

The mixtures prepared according to the invention are advantageously polymer formulations.

Polymer formulations are mixtures of a polymer with one or more other polymers and/or with organic and/or inorganic additives.

The additives may be liquid or solid, and their processing properties may vary widely.

Examples of processing properties are viscosity, density or, in the case of liquids, surface tension, or, in the case of solid additives, grain size, grain shape, grain size distribution, hardness, flowability, adhesion, or bulk density.

The additives give the polymer formulations the properties demanded by the respective application.

Examples which may be mentioned of the large number of additives known in the prior art are fillers, which may be used in the form of beads, fibers, or lamellae, with dimensions of from 10 nm to a few millimeters. They are used mainly to adjust the mechanical properties of the polymer formulations.

Examples of other additives are light stabilizers, in particular stabilizers to prevent damage by UV and visible light, flame retardants, processing aids, pigments, lubricants and friction additives, coupling agents, impact modifiers, flow agents, mold-release agents, nucleating agents, acid scavengers, base scavengers, antioxidants.

These additives for plastics are described by way of example by H. Zweifel in: Plastics Additives Handbook, 5th edition, Hanser Verlag 2000, incorporated herein by way of reference.

Other additives which may be used are thermoplastic and/or non-thermoplastic polymers, in particular thermoplastic polymers, thus preparing blends and polymer alloys with concentration gradients.

For the purposes of the invention, the term polymers fundamentally includes all of the known, synthetic, naturally occurring, and modified naturally occurring polymers, i.e. thermoplastic or thermoset polymers, including elastomeric polymers.

Examples of thermoset polymers are epoxy resins, phenolic resins, or alkyd resins.

It is particularly preferable to use thermoplastic polymers which can be processed by melt extrusion.

By way of example, mention may be made of:

polylactones, such as poly(pivalolactone), poly(caprolactone);

polyurethanes, such as the polymerization products of the diisocyanates, e.g. of naphthalene 1,5-diisocyanate; p-phenylene diisocyanates; m-phenylene diisocyanate, tolylene 2,4-diisocyanate, tolylene 2,6-diisocyanate, diphenylmethane 4,4′-diisocyanate, 3,3′-dimethylbiphenyl 4,4′-diisocyanate, diphenylisopropylidene 4,4′-diisocyanate, 3,3′-dimethyldiphenyl 4,4′-diisocyanate, 3,3′-dimethyldiphenylmethane 4,4′-diisocyanate, 3,3′-dimethoxybiphenyl 4,4′-diisocyanate, dianisidine diisocyanate, toluidine diisocyanate, hexamethylene diisocyanate, 4,4′-diisocyanatodiphenylmethane, hexamethylene 1,6-diisocyanate, or dicyclohexylmethane 4,4′-diisocyanate, with long-chain diols, for example with poly(tetramethylene adipate), poly(ethylene adipate), poly(butylene 1,4-adipate), poly(ethylene succinate), poly(butylene 2,3-succinate), with polyether diols, and/or with one or more diols such as ethylene glycol, propylene glycol, and/or with a polydiol, such as diethylene glycol, triethylene glycol, and/or tetraethylene glycol;

polycarbonates, such as poly[methanebis(phenyl 4-carbonate)], poly[1,1-etherbis(phenyl 4-carbonate)], poly[diphenylmethanebis(phenyl 4-carbonate)], poly[1,1-cyclohexanebis(phenyl carbonate)];

polysulfones, such as the reaction product of the sodium salt of 2,2-bis(4-hydroxyphenyl)propane or of 4,4′-dihydroxydiphenyl ether with 4,4′-dichlorodiphenyl sulfone;

polyethers, polyketones, and polyether ketones, such as polymerization products of hydroquinone, of 4,4′-dihydroxybiphenyl, of 4,4′-dihydroxybenzophenone, or of 4,4′-dihydroxydiphenylsulfone with dihalogenated, in particular difluorinated or dichlorinated, aromatic compounds of the type represented by 4,4′-dihalodiphenyl sulfone, 4,4′-dihalodibenzophenone, bis(4,4′-dihalobenzoyl)benzene, 4,4′-dihalobiphenyl;

polyamides, such as poly(4-aminobutanoic acid), poly(hexamethyleneadipamide), poly(6-aminohexanoic acid), poly(m-xylyleneadipamide), poly(p-xylylenesebacamide), poly(2,2,2-trimethylhexamethyleneterephthalamide), poly(meta-phenyleneisophthalamide) (NOMEX), poly(p-phenyleneterephthalamide) (KEVLAR);

polyesters, such as poly(ethylene acetate), poly(ethylene 1,5-naphthalate), poly(cyclohexane-1,4-dimethylene terephthalate), poly(ethylene oxybenzoate) (A-TELL), poly(parahydroxybenzoate) (EKONOL), poly(cyclohexylidene-1,4-dimethylene terephthalate) (KODEL), polyethylene terephthalate, polybutylene terephthalate;

poly(arylene oxides), such as poly(2,6-dimethylphenylene 1,4-oxide), poly(2,6-diphenylphenylene 1,4-oxide);

homo- and copolyacetals, such as oxymethylene polymers;

liquid-crystalline polymers, such as the polycondensation products from the group of monomers consisting of terephthalic acid, isophthalic acid, naphthalene-1,4-carboxylic acid, naphthalene-2,6-dicarboxylic acid, biphenyl-4,4′-dicarboxylic acid, 4-hydroxybenzoic acid, 6-hydroxy-2-naphthalenedicarboxylic acid, hydroquinone, 4,4′-dihydroxybiphenyl, 4-aminophenol;

poly(arylene sulfides), such as poly(phenylene sulfide), poly(phenylene sulfide ketone), poly(phenylene sulfide sulfone);

polyetherimides;

vinyl polymers and their copolymers, such as polyvinyl acetate, polyvinyl chloride, polyvinyl butyral, polyvinylidene chloride, ethylene-vinyl acetate copolymers;

polyacrylic derivatives, such as polyacrylate and its copolymers, e.g. polyethyl acrylate, poly(n-butyl acrylate), polymethyl methacrylate, polyethyl methacrylate, poly(n-butyl methacrylate), poly(n-propyl methacrylate), polyacrylonitrile, water-insoluble ethylene-acrylic acid copolymers, water-insoluble ethylene-vinyl alcohol copolymers, acrylonitrile copolymers, methyl methacrylate-styrene copolymers, ethylene-ethyl acrylate copolymers, acrylonitrile-butadiene-styrene copolymers;

polyolefins, such as polyethylene, in particular high-density and low-density poly(ethylene), polypropylene, chlorinated low-density poly(ethylene), poly(4-methyl-1-pentene), poly(styrene);

water-insoluble ionomers;

poly(epichlorohydrin);

furan polymers, such as poly(furan);

cellulose esters, such as cellulose acetate, cellulose acetate butyrate, cellulose propionate;

silicones, such as poly(dimethylsiloxane), poly(dimethylsiloxane-co-phenylmethylsiloxane);

protein thermoplastics;

and also all of the mixtures and alloys (miscible and immiscible blends) of two or more of the polymers mentioned.

For the purposes of the invention, polymers also encompass elastomers derived, for example, from one or more of the following polymers:

brominated butyl rubber, chlorinated butyl rubber, polyurethane elastomers, fluoroelastomers, polyester elastomers, elastomeric polyvinyl chloride, butadiene-acrylonitrile elastomers, silicone elastomers, poly(butadiene), poly(isobutylene), ethylene-propylene copolymers, ethylene-propylene-diene terpolymers, sulfonated ethylene-propylene-diene terpolymers, poly(chloroprene), poly(2,3-dimethylbutadiene), poly(butadiene-pentadiene), chlorosulfonated poly(ethylenes), poly(sulfide) elastomers, block copolymers, built up from segments of amorphous or of (semi)crystalline blocks, such as poly(styrene), poly(vinyltoluene), poly(tert-butylstyrene), polyesters, and the like, and of elastomeric blocks, such as poly(butadiene), poly(isoprene), ethylene-propylene copolymers, ethylene-butylene copolymers, ethylene-isoprene copolymers, and hydrogenated derivatives of these, e.g. SEBS, SEPS, SEEPS, and also hydrogenated ethylene-isoprene copolymers with a relatively high proportion of 1,2-linked isoprene, polyethers, such as the products marketed by Kraton Polymers with the trade name KRATON®.

1. A process for the continuous preparation of mixtures from at least two components, encompassing the steps of:
a) charging the individual components to storage vessels,
b) introducing each individual component by way of a conveying device for that component into a mixing device,
c) varying the conveying rate of at least one conveying device in such a way that this conveying rate varies periodically between a lower and an upper limiting value, and
d) mixing the individual components in the mixing device.

2. The process as claimed in claim 1, wherein the variation of the conveying rate of one conveying device continuously rises or falls, and wherein the variation in the conveying rate of all of the other conveying devices is periodic.

3. The process as claimed in claim 1, wherein the variation of the conveying rate of various conveying devices is periodic and wherein the frequencies of the variations differ from one another.

4. The process as claimed in claim 1, wherein the variation of the conveying rate of at least one conveying device, preferably of all of the periodic variations, corresponds to a sawtooth function or a sine function, the periods thereof preferably being constant over time.

5. The process as claimed in claim 1, wherein the variation of the conveying rate of at least one conveying device corresponds to a periodic step function whose periods and step intervals are preferably constant over time.

6. The process as claimed in claim 1, wherein the periods or step intervals for the variation of the conveying rates of the individual conveying devices are an integral multiple of a base period, where the ratio of any two desired periods or step intervals for the variation of the conveying rate of the conveying devices, or the ratio of a period and a step interval for the variation of the conveying rate of two conveying devices, is preferably equal to half of a whole number, and is in particular 0.5, or 1.5, or 2.5.

7. The process as claimed in claim 1, wherein the frequency ratio of two periodic variations of the conveying rate of two conveying devices is proportional to the compositional resolution desired.

8. The process as claimed in claim 1, wherein the total conveying rate of all of the conveying devices is constant over time.

9. The process as claimed in claim 1, wherein at least one component is a liquid, a conveyable solid, and/or a gas.

10. The process as claimed in claim 9, wherein at least one component is a polymer melt, and wherein at least one other component is an additive.

11. An apparatus for carrying out the process as claimed in claim 1, encompassing the following units:
i) storage vessels for each individual component of the mixture to be prepared,
ii) mixing device for mixing all of the components of the mixture to be prepared;
iii) lines for the individual components, leading from each individual storage vessel to the mixing device;
iv) in every line for every individual component, conveying devices whose conveying rate can be set individually; and
v) control device for the conveying devices, which controls the conveying rate of each conveying device independently of the others, and which sets the conveying rate of at least one conveying device variably and periodically between a predetermined lower limiting value and a predetermined upper limiting value.

12. The apparatus as claimed in claim 11, wherein the mixing device is a static mixer.

13. The apparatus as claimed in claim 11, wherein the mixing device is a screw extruder.

14. The use of the process as claimed in claim 1 for producing substance libraries for high-throughput screening and other combinatorial methods.

15. The use as claimed in claim 14, wherein moldings are produced, preferably in the form of film strips, extrudates, or pellets produced from these extrudates.

16. The use as claimed in claim 15, wherein the molding is an extrudate or an unsupported film strip, from which discrete fractions are produced by chopping or stamping, or by pelletizing.
